SEQUENCE LISTING (27 sequences)

SEQ ID NO: 1 - DNA, 2803 bp, Zea mays
  source 1..2803: mol_type = genomic DNA
actatgacccctgctgcctttccaccttcctcgatctccctagctcgctctccaatctcccatttctctctctctctctctctctctctctctctcgtcttcgccatccaccatccatcccgttcgtcgtgatagatggcggcagtccagccgtccgtggagatgacgacgcagcaagggccgccagccgccagctattctccggccagggtacgtcgtcggtcgtcgtccgtcaccaatcggactccttagtaatattggcagacacgcaacgcaataagaacaagtcgatctcgatcgcatatgcatgcaggatggcggcggcggcaggagcgccgaggatgaggagaaggagaaggcggcggcgatcgacaactggcttcccatcagcgcgacgcggaatgccaagtggtggtactccgccttccacaacgtcacggccatggtcggcgccggcgtgctcggcctcccctacgccatgtcccagctcggctggtacgcacgcatcaatcaatcagtcatcatttccattctgatgataggagtacttgtcatgaatgtaactactgctgtggattcaggggtgccggcatcacgatcatgctcctgtcgtgggtgatcacgctgtacacgctgtggcagatggtggagatgcacgagatggtgccggggaagcggttcgaccggtaccacgagctggggcagcacgcgttcggggaccggctgggcctgtggatcgtggtgccgcagcagctggtggtggaggtgggggtgaacatcgtgtacatggtgaccggcggcacgtcgctgcagaagttccacgacgtgctggtgtgcggcgacgcggcgtgcgagggcgggcgcaagatcaggctcacctacttcatcatgatcttcgcgtcctgccacttcgtgctggcgcagctgcccaacttcgactccatctccggcgtgtccctggccgccgccgtcatgtcgctcagctactcgaccatcgcgtggggcgcgtccgtcagcaaagggcgggtgcccgacgtggactacggactcagggccaccacgccgccgggcaaggtgttcgggttcctgggcgcgctcgggaccgtggcgttcgcctacgcgggccacaacgtggtgctggagatccaggccaccatcccgtccactcccgagaagccgtccaagaagcccatgtggaagggcgtcgtcgtcgcctacctcgtcgtcgcgctctgctacttccccgtctcgttcgtcggctactgggctttcggcgatagcgtcgacggcgacatcctcgtcacccttaacagacccaggtggctcatcgccctcgccaacatgatggtcgtcatccatgtcattggcagctaccaggtaattaagtcaaactaattattaagctagcagctttagcatctctgaactgatcaatgcttgtgtttggctgcacacagatctatgcgatgcccgtgttcgacatgatagagacggtgctggtgaagaagctgcgcttccctccaggcctcacgctccgtctcattgcccgtactgtctacgttggtacgttgtctgaaacttttcagtccaaatgagcctttacaaatgtgaggaacaaaaccagactgacctgacgagatcgatgccacaacgtgtgctgcagcgttcacaatgttcatagccatcacgtttcccttcttcgacgggctgctcagcttcttcggcggattcgcgttcgcgcccacaacctatttcgtaagctcctgctgcacctgcactgtcgttttaggccggggtttcagaattgcaacaagactactacatgagagctagctgaccaaatcttcttgcttttcttcttcttctgctgccagcttccatgcatcatgtggctcgcaatctacaagcccaaacggttcagcctctcatggttcaccaactgggtaagaagaagagccaaccaaataccccaccaatgcaatccaacagcagcctatctaattaactctaacctactcgtgtgctcttcctcttttatgttcttcagatctgcatcatccttggagtgcttctgatggtgctggcgccgattggagggctccggcagatcattatcagcgccaagacatacaagttctaccagtaaacattgcacaattatcgtgcaaaaaacaacgcaaaccccttgcttcaccagtaactgacagacaagtcttgttaggccatatataccatgttaccatcagctgaacaggttctggattttgttccatgagggcttccaactccttgtttaatttggcacgacaaaaaactcgcaccgggtgctctaatcggcggtcgattacagcgtccagtgcgagttctttgccgtgccaaaattgaaaggaggtcggctcggatgccggggcatgcatacaggacctagaacccgtttcggttgatgagaatgttcctggcatgtaatgcttcccttgttggttgtggtctaacctaggagtttgctttgagtggatctttgtacaattgtacgtgtatatgatgtttttttacattattcaggggaattggatcttagcttctgtgtgtgtatgcgtaaaaagggaaagagttgtgcttcatatatgtatatttttagatggcctaaaatgtgtcgtatttgttgcatttttttttctcctaccacttttcattcaattaatcacgaaaaaaatacattgatgatcatgcgttgttgtacttgatggatcttgataaacagtgggtgcattggtacagtttagatgcacggtgaatatttggt

SEQ ID NO: 2 - DNA, 1419 bp, synthetic construct
  source 1..1419: mol_type = other DNA
atggcggcagtccagccgtccgtggagatgacgacgcagcaagggccgccagccgccagctattctccggccagggatggcggcggcggcaggagcgccgaggatgaggagaaggagaaggcggcggcgatcgacaactggcttcccatcagcgcgacgcggaatgccaagtggtggtactccgccttccacaacgtcacggccatggtcggcgccggcgtgctcggcctcccctacgccatgtcccagctcggctggggtgccggcatcacgatcatgctcctgtcgtgggtgatcacgctgtacacgctgtggcagatggtggagatgcacgagatggtgccggggaagcggttcgaccggtaccacgagctggggcagcacgcgttcggggaccggctgggcctgtggatcgtggtgccgcagcagctggtggtggaggtgggggtgaacatcgtgtacatggtgaccggcggcacgtcgctgcagaagttccacgacgtgctggtgtgcggcgacgcggcgtgcgagggcgggcgcaagatcaggctcacctacttcatcatgatcttcgcgtcctgccacttcgtgctggcgcagctgcccaacttcgactccatctccggcgtgtccctggccgccgccgtcatgtcgctcagctactcgaccatcgcgtggggcgcgtccgtcagcaaagggcgggtgcccgacgtggactacggactcagggccaccacgccgccgggcaaggtgttcgggttcctgggcgcgctcgggaccgtggcgttcgcctacgcgggccacaacgtggtgctggagatccaggccaccatcccgtccactcccgagaagccgtccaagaagcccatgtggaagggcgtcgtcgtcgcctacctcgtcgtcgcgctctgctacttccccgtctcgttcgtcggctactgggctttcggcgatagcgtcgacggcgacatcctcgtcacccttaacagacccaggtggctcatcgccctcgccaacatgatggtcgtcatccatgtcattggcagctaccagatctatgcgatgcccgtgttcgacatgatagagacggtgctggtgaagaagctgcgcttccctccaggcctcacgctccgtctcattgcccgtactgtctacgttgcgttcacaatgttcatagccatcacgtttcccttcttcgacgggctgctcagcttcttcggcggattcgcgttcgcgcccacaacctatttccttccatgcatcatgtggctcgcaatctacaagcccaaacggttcagcctctcatggttcaccaactggatctgcatcatccttggagtgcttctgatggtgctggcgccgattggagggctccggcagatcattatcagcgccaagacatacaagttctaccagtaa

SEQ ID NO: 3 - protein, 472 aa, Zea mays
MAAVQPSVEMTTQQGPPAASYSPARDGGGGRSAEDEEKEKAAAIDNWLPISATRNAKWWYSAFHNVTAMVGAGVLGLPYAMSQLGWGAGITIMLLSWVITLYTLWQMVEMHEMVPGKRFDRYHELGQHAFGDRLGLWIVVPQQLVVEVGVNIVYMVTGGTSLQKFHDVLVCGDAACEGGRKIRLTYFIMIFASCHFVLAQLPNFDSISGVSLAAAVMSLSYSTIAWGASVSKGRVPDVDYGLRATTPPGKVFGFLGALGTVAFAYAGHNVVLEIQATIPSTPEKPSKKPMWKGVVVAYLVVALCYFPVSFVGYWAFGDSVDGDILVTLNRPRWLIALANMMVVIHVIGSYQIYAMPVFDMIETVLVKKLRFPPGLTLRLIARTVYVAFTMFIAITFPFFDGLLSFFGGFAFAPTTYFLPCIMWLAIYKPKRFSLSWFTNWICIILGVLLMVLAPIGGLRQIIISAKTYKFYQ

SEQ ID NO: 4 - DNA, 36 bp, synthetic construct
  source 1..36: mol_type = other DNA
aataatggtctcaggcgggagatgacgacgcagcaa

SEQ ID NO: 5 - DNA, 36 bp, synthetic construct
  source 1..36: mol_type = other DNA
attattggtctctaaacgagaatagctggcggctgg

SEQ ID NO: 6 - DNA, 41 bp, synthetic construct
  source 1..41: mol_type = other DNA
gggagatgacgacgcagcaagttttagagctagaaatagca

SEQ ID NO: 7 - DNA, 35 bp, synthetic construct
  source 1..35: mol_type = other DNA
gagaatagctggcggctggcgcttcttggtgccgc

SEQ ID NO: 8 - DNA, 22 bp, synthetic construct
  source 1..22: mol_type = other DNA
ttaacaaaccaagctgagatgc

SEQ ID NO: 9 - DNA, 18 bp, synthetic construct
  source 1..18: mol_type = other DNA
cagcacgaagtggcagga

SEQ ID NO: 10 - DNA, 19 bp, synthetic construct
  source 1..19: mol_type = other DNA
cgctctccaatctcccatt

SEQ ID NO: 11 - DNA, 19 bp, synthetic construct
  source 1..19: mol_type = other DNA
acggcaaagaactcgcact

SEQ ID NO: 12 - DNA, 19 bp, synthetic construct
  source 1..19: mol_type = other DNA
tactgggctttcggcgata

SEQ ID NO: 13 - DNA, 20 bp, synthetic construct
  source 1..20: mol_type = other DNA
tgaacattgtgaacgcaacg

SEQ ID NO: 14 - DNA, 23 bp, synthetic construct
  source 1..23: mol_type = other DNA
tggagatgacgacgcagcaaggg

SEQ ID NO: 15 - DNA, 23 bp, synthetic construct
  source 1..23: mol_type = other DNA
gccagccgccagctattctccgg

SEQ ID NO: 16 - DNA, 1421 bp, synthetic construct
  source 1..1421: mol_type = other DNA
atggcggcagtccagccgtccgtggagatgacgacgcagacaagggccgccagccgccagctatttctccggccagggatggcggcggcggcaggagcgccgaggatgaggagaaggagaaggcggcggcgatcgacaactggcttcccatcagcgcgacgcggaatgccaagtggtggtactccgccttccacaacgtcacggccatggtcggcgccggcgtgctcggcctcccctacgccatgtcccagctcggctggggtgccggcatcacgatcatgctcctgtcgtgggtgatcacgctgtacacgctgtggcagatggtggagatgcacgagatggtgccggggaagcggttcgaccggtaccacgagctggggcagcacgcgttcggggaccggctgggcctgtggatcgtggtgccgcagcagctggtggtggaggtgggggtgaacatcgtgtacatggtgaccggcggcacgtcgctgcagaagttccacgacgtgctggtgtgcggcgacgcggcgtgcgagggcgggcgcaagatcaggctcacctacttcatcatgatcttcgcgtcctgccacttcgtgctggcgcagctgcccaacttcgactccatctccggcgtgtccctggccgccgccgtcatgtcgctcagctactcgaccatcgcgtggggcgcgtccgtcagcaaagggcgggtgcccgacgtggactacggactcagggccaccacgccgccgggcaaggtgttcgggttcctgggcgcgctcgggaccgtggcgttcgcctacgcgggccacaacgtggtgctggagatccaggccaccatcccgtccactcccgagaagccgtccaagaagcccatgtggaagggcgtcgtcgtcgcctacctcgtcgtcgcgctctgctacttccccgtctcgttcgtcggctactgggctttcggcgatagcgtcgacggcgacatcctcgtcacccttaacagacccaggtggctcatcgccctcgccaacatgatggtcgtcatccatgtcattggcagctaccagatctatgcgatgcccgtgttcgacatgatagagacggtgctggtgaagaagctgcgcttccctccaggcctcacgctccgtctcattgcccgtactgtctacgttgcgttcacaatgttcatagccatcacgtttcccttcttcgacgggctgctcagcttcttcggcggattcgcgttcgcgcccacaacctatttccttccatgcatcatgtggctcgcaatctacaagcccaaacggttcagcctctcatggttcaccaactggatctgcatcatccttggagtgcttctgatggtgctggcgccgattggagggctccggcagatcattatcagcgccaagacatacaagttctaccagtaa

SEQ ID NO: 17 - DNA, 1411 bp, synthetic construct
  source 1..1411: mol_type = other DNA
atggcggcagtccagccgtccgtggagatgacgacgcaagggccgccagccgccagctccggccagggatggcggcggcggcaggagcgccgaggatgaggagaaggagaaggcggcggcgatcgacaactggcttcccatcagcgcgacgcggaatgccaagtggtggtactccgccttccacaacgtcacggccatggtcggcgccggcgtgctcggcctcccctacgccatgtcccagctcggctggggtgccggcatcacgatcatgctcctgtcgtgggtgatcacgctgtacacgctgtggcagatggtggagatgcacgagatggtgccggggaagcggttcgaccggtaccacgagctggggcagcacgcgttcggggaccggctgggcctgtggatcgtggtgccgcagcagctggtggtggaggtgggggtgaacatcgtgtacatggtgaccggcggcacgtcgctgcagaagttccacgacgtgctggtgtgcggcgacgcggcgtgcgagggcgggcgcaagatcaggctcacctacttcatcatgatcttcgcgtcctgccacttcgtgctggcgcagctgcccaacttcgactccatctccggcgtgtccctggccgccgccgtcatgtcgctcagctactcgaccatcgcgtggggcgcgtccgtcagcaaagggcgggtgcccgacgtggactacggactcagggccaccacgccgccgggcaaggtgttcgggttcctgggcgcgctcgggaccgtggcgttcgcctacgcgggccacaacgtggtgctggagatccaggccaccatcccgtccactcccgagaagccgtccaagaagcccatgtggaagggcgtcgtcgtcgcctacctcgtcgtcgcgctctgctacttccccgtctcgttcgtcggctactgggctttcggcgatagcgtcgacggcgacatcctcgtcacccttaacagacccaggtggctcatcgccctcgccaacatgatggtcgtcatccatgtcattggcagctaccagatctatgcgatgcccgtgttcgacatgatagagacggtgctggtgaagaagctgcgcttccctccaggcctcacgctccgtctcattgcccgtactgtctacgttgcgttcacaatgttcatagccatcacgtttcccttcttcgacgggctgctcagcttcttcggcggattcgcgttcgcgcccacaacctatttccttccatgcatcatgtggctcgcaatctacaagcccaaacggttcagcctctcatggttcaccaactggatctgcatcatccttggagtgcttctgatggtgctggcgccgattggagggctccggcagatcattatcagcgccaagacatacaagttctaccagtaa

SEQ ID NO: 18 - DNA, 1394 bp, synthetic construct
  source 1..1394: mol_type = other DNA
atggcggcagtccagccgtccgtggagatgacgacgcagctccggccagggatggcggcggcggcaggagcgccgaggatgaggagaaggagaaggcggcggcgatcgacaactggcttcccatcagcgcgacgcggaatgccaagtggtggtactccgccttccacaacgtcacggccatggtcggcgccggcgtgctcggcctcccctacgccatgtcccagctcggctggggtgccggcatcacgatcatgctcctgtcgtgggtgatcacgctgtacacgctgtggcagatggtggagatgcacgagatggtgccggggaagcggttcgaccggtaccacgagctggggcagcacgcgttcggggaccggctgggcctgtggatcgtggtgccgcagcagctggtggtggaggtgggggtgaacatcgtgtacatggtgaccggcggcacgtcgctgcagaagttccacgacgtgctggtgtgcggcgacgcggcgtgcgagggcgggcgcaagatcaggctcacctacttcatcatgatcttcgcgtcctgccacttcgtgctggcgcagctgcccaacttcgactccatctccggcgtgtccctggccgccgccgtcatgtcgctcagctactcgaccatcgcgtggggcgcgtccgtcagcaaagggcgggtgcccgacgtggactacggactcagggccaccacgccgccgggcaaggtgttcgggttcctgggcgcgctcgggaccgtggcgttcgcctacgcgggccacaacgtggtgctggagatccaggccaccatcccgtccactcccgagaagccgtccaagaagcccatgtggaagggcgtcgtcgtcgcctacctcgtcgtcgcgctctgctacttccccgtctcgttcgtcggctactgggctttcggcgatagcgtcgacggcgacatcctcgtcacccttaacagacccaggtggctcatcgccctcgccaacatgatggtcgtcatccatgtcattggcagctaccagatctatgcgatgcccgtgttcgacatgatagagacggtgctggtgaagaagctgcgcttccctccaggcctcacgctccgtctcattgcccgtactgtctacgttgcgttcacaatgttcatagccatcacgtttcccttcttcgacgggctgctcagcttcttcggcggattcgcgttcgcgcccacaacctatttccttccatgcatcatgtggctcgcaatctacaagcccaaacggttcagcctctcatggttcaccaactggatctgcatcatccttggagtgcttctgatggtgctggcgccgattggagggctccggcagatcattatcagcgccaagacatacaagttctaccagtaa

SEQ ID NO: 19 - DNA, 25 bp, synthetic construct
  source 1..25: mol_type = other DNA
aagggccgccagccgccagctattc

SEQ ID NO: 20 - DNA, 61 bp, synthetic construct
  source 1..61: mol_type = other DNA
cgtccgtggagatgacgacgcagcaagggccgccagccgccagctattctccggccagggt

SEQ ID NO: 21 - DNA, 63 bp, synthetic construct
  source 1..63: mol_type = other DNA
cgtccgtggagatgacgacgcagacaagggccgccagccgccagctatttctccggccagggt

SEQ ID NO: 22 - DNA, 53 bp, synthetic construct
  source 1..53: mol_type = other DNA
cgtccgtggagatgacgacgcaagggccgccagccgccagctccggccagggt

SEQ ID NO: 23 - DNA, 36 bp, synthetic construct
  source 1..36: mol_type = other DNA
cgtccgtggagatgacgacgcagctccggccagggt

SEQ ID NO: 24 - DNA, 39 bp, synthetic construct
  source 1..39: mol_type = other DNA
cgacgcagcaagggccgccagccgccagctattctccgg

SEQ ID NO: 25 - DNA, 41 bp, synthetic construct
  source 1..41: mol_type = other DNA
cgacgcagacaagggccgccagccgccagctatttctccgg

SEQ ID NO: 26 - DNA, 31 bp, synthetic construct
  source 1..31: mol_type = other DNA
cgacgcaagggccgccagccgccagctccgg

SEQ ID NO: 27 - DNA, 14 bp, synthetic construct
  source 1..14: mol_type = other DNA
cgacgcagctccgg

(19) United States
(12) Patent Application Publication
(10) Pub. No.: US 2026/0242813 A1
(43) Pub. Date: Aug. 20, 2026

(54) USE OF ZEA MAYS AMINO ACID TRANSPORTER AND CODING GENE THEREOF IN PLANT DISEASE RESISTANCE

(71) Applicant: NORTHWEST A & F UNIVERSITY, Xianyang (CN)

(72) Inventors: Qin YANG, Xianyang (CN); Yapeng WANG, Xianyang (CN); Zhe WANG, Xianyang (CN); Huanhuan TAI, Xianyang (CN); Xiaojian HUANG, Xianyang (CN); Huiqin NIAN, Xianyang (CN)

(73) Assignee: NORTHWEST A & F UNIVERSITY, Xianyang (CN)

(21) Appl. No.: 19/493,626

(22) PCT Filed: Oct. 31, 2023
(86) PCT No.: PCT/CN2023/128135

§ 371 Date: Dec. 16, 2025

(30) Foreign Application Priority Data
Nov. 7, 2022 (CN) 202211382642.3

(51) Int. Cl. C12N 15/82; C07K 14/415; C12N 9/22; C12N 15/11

(52) U.S. Cl. CPC C12N 15/8251; C07K 14/415; C12N 9/226; C12N 15/111; C12N 15/8282; C12N 2310/20

(57) ABSTRACT

Provided is use of a Zea mays amino acid transporter and a coding gene thereof in plant disease resistance. Specifically provided are a protein having the amino acid sequence of SEQ ID NO: 3 and a coding gene thereof. A ZmLHT1 gene is knocked out based on CRISPR-Cas9 system to obtain a ZmLHT1 mutant and the disease attack level of the mutant in the filed is further evaluated by means of disease resistance identification. The result shows that the ZmLHT1 mutant has a disease attack level significant higher than that of wild-type Zea mays and allows the Zea mays yield to be significantly increased in the presence of southern leaf blight. The ZmLHT1 gene and the coding gene thereof can regulate the disease resistance of plants, and the resistance of Zea mays to southern leaf blight can be significantly enhanced by means of reducing content and/or activity of ZmLHT1 proteins.

### CROSS REFERENCE TO THE RELATED APPLICATIONS

This application is the national phase entry of International Application No. PCT/CN2023/128135, filed on Oct. 31, 2023, which is based upon and claims priority to Chinese Patent Application No. 202211382642.3, filed on Nov. 7, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named KJ0123S_Sequence_Listing.xml, created on Oct. 22, 2025, and is 33,678 bytes in size.

### TECHNICAL FIELD

The present invention belongs to the field of plant genetic engineering technology, and specifically relates to use of a maize amino acid transporter protein and coding gene thereof in plant disease resistance.

### BACKGROUND

Maize (Zea mays) is one of the most important cereal crops in the world, has a wide range of uses. It plays a crucial role in maintaining food security, promoting livestock development, and meeting industrial raw material needs and so on. In recent years, southern leaf blight has frequently occurred during maize planting process, leading to a sharp decline in maize yield and seriously affecting the normal development of the grain industry. Southern leaf blight (SLB) is a saprophytic leaf fungal disease caused by Bipolaris maydis in maize, and is the most prevalent disease in summer maize production areas in China. At present, the prevention and control of southern leaf blight in China mainly rely on chemical control and planting resistant varieties. The extensive use of chemical fungicides can cause a series of problems such as ecological environment pollution and food safety. Cultivating maize varieties resistant to southern leaf blight is the most economical and effective way to reduce the impact of southern leaf blight on maize yield.

Therefore, digging and identifying disease resistance related genes can provide important genetic resources for cultivating new varieties of disease resistant plants, which fundamentally prevents and controls the harm of maize southern leaf blight, promotes the commercial maize breeding process, and is of significance for ensuring the quality and high and stable yield of maize, having a wide range of application value in the field of maize molecular breeding.

### SUMMARY

The technical problem to be solved by the present invention is how to regulate disease resistance of plants, and/or, how to improve disease resistance of plants. The technical problem to be solved is not limited to the described technical subject, and the skilled in the art can clearly understand other technical subjects not mentioned herein through the following description.

To solve the above technical problems, the present invention first provides use of protein(s) or substance(s) that regulate activity and/or content of the proteins, the use can be any one of the followings:
A1) use of proteins or substances regulating activity and/or content of the proteins in regulating disease resistance of plant;
A2) use of proteins or substances regulating activity and/or content of the proteins in the preparation of products that regulate disease resistance of plant;
A3) use of proteins or substances regulating activity and/or content of the proteins in cultivating disease resistant plants;
A4) use of proteins or substances regulating activity and/or content of the proteins in the preparation of products for cultivating disease resistant plants;
A5) use of proteins or substances regulating activity and/or content of the proteins in plant breeding or improvement of plant germplasm resources;
the protein has a name of ZmLHT1, which can be any one of the following:
B1) a protein having an amino acid sequence of SEQ ID NO: 3;
B2) a protein obtained by substituting and/or deleting and/or adding amino acid residue(s) to an amino acid sequence shown in SEQ ID NO: 3, which has 80% or more identity with and the same function as the protein in B1);
B3) a fusion protein which is obtained by linking tag(s) at the N-terminus and/or C-terminus of B1) or B2) and has the same function.

In the above uses, the protein ZmLHT1 can be derived from maize.

Furthermore, the protein ZmLHT1 can be an amino acid transporter, which has amino acid transport function.

Furthermore, the protein ZmLHT1 can be a maize disease resistance related protein, specifically a maize southern leaf blight resistance related protein.

In order to facilitate purification or detection of the protein in B1), a tag protein can be linked to the amino or carboxyl terminus of a protein composed of an amino acid sequence shown in SEQ ID NO: 3 in the sequence listing.

The tag protein includes but is not limited to: GST (glutathione S-transferase) tag protein, His6 tag protein (His-tag), MBP (maltose binding protein) tag protein, Flag tag protein, SUMO tag protein, HA tag protein, Myc tag protein, eGFP (enhanced green fluorescent protein), eCFP (enhanced cyan fluorescent protein), eYFP (enhanced yellow green fluorescent protein), mCherry (monomeric red fluorescent protein) or AviTag tag protein.

Ordinary technicians in the art can easily use known methods, such as directed evolution or point mutation, to mutate a nucleotide sequence encoding protein ZmLHT1 of the present invention. Those nucleotides that have been artificially modified and have 75% or more identity with a nucleotide sequence of a protein ZmLHT1 isolated in the present invention, as long as they encode the protein ZmLHT1 and have the function of the protein ZmLHT1, are sequences derived from the nucleotide sequence of the present invention and equivalent to the sequence of the present invention.

The above 75% or more identity can be 80%, 85%, 90%, or 95% or more identity.

Herein, identity refers to the identity of amino acid or nucleotide sequences. Homology search sites on the international Internet can be used to determine the identity of amino acid sequences, such as BLAST page of the NCBI homepage website. For example, in advanced BLAST2.1, by using blastp as a program, setting Expect value to 10, setting all Filters to OFF, using BLOSUM62 as Matrix, and setting Gap existence cost, Per residue gap cost, and Lambda ratio to 11, 1, and 0.85 (default values), respectively, retrieval can be performed to calculate identity of amino acid sequences, and then an identity value (%) can be obtained.

Herein, the above 80% and more identity can be at least 80%, 81%, 82%, 83%, 84%, 85%, 86%, 87%, 88%, 89%, 90%, 91%, 92%, 93%, 94%, 95%, 96%, 97%, 98% or 99% identity.

Herein, the substances regulating activity and/or content of the proteins can be a substance regulating gene expression, and the gene encodes the protein ZmLHT1.

In the above, the substance that regulates gene expression can be a substance that performs at least one of the following six types of regulations: 1) regulation on transcriptional level of the gene; 2) regulation on post-transcription of the gene (including regulation of modifications, splicing, and/or processing of the transcription product of the gene); 3) regulation on RNA transport of the gene (including regulation on mRNA transport from nucleus to cytoplasm); 4) regulation on translation of the gene; 5) regulation on mRNA degradation of the gene; 6) regulation on post translation of the gene (including regulation on activity of a protein translated by the gene, such as regulation of protein precursor processing, protein transport, protein degradation, and/or protein folding).

The substance regulating gene expression can specifically be any one of the biomaterials described in E1)-E4) herein.

Further, the substance regulating gene expression may be a substance (including nucleic acid molecules or vectors) that inhibits, reduces, or downregulates expression of a coding gene of the protein ZmLHT1. The substance that inhibits, reduces, or downregulates expression of a coding gene of the protein ZmLHT1 can be an agent for knocking out the gene (ZmLHT1 gene), such as an agent for knocking out the gene through CRISPR-Cas9, or a reagent for knocking out the gene through homologous recombination. The agent that inhibits or reduces the gene expression may include a polynucleotide targeting the gene, such as siRNA, shRNA, sgRNA, miRNA, or antisense RNA.

Furthermore, the substance regulating gene expression can also be a substance (including nucleic acid molecules or vectors) that enhances or upregulates expression of a coding gene of the protein ZmLHT1.

The present invention also provides a use of biomaterials related to the protein ZmLHT1, the use can be any one of the followings:
D1) use of biomaterials related to the protein ZmLHT1 in regulating plant disease resistance;
D2) use of biomaterials related to the protein ZmLHT1 in the preparation of products regulating plant disease resistance;
D3) use of biomaterials related to the protein ZmLHT1 in cultivating disease resistant plants;
D4) use of biomaterials related to the protein ZmLHT1 in the preparation of products for cultivating disease resistant plants;
D5) use of biomaterials related to the protein ZmLHT1 in plant breeding or improvement of plant germplasm resources;
the biomaterial can be any one of the following E1) to E6):
E1) a nucleic acid molecule encoding the protein ZmLHT1;
E2) a nucleic acid molecule that inhibits or reduces expression of a coding gene of the protein ZmLHT1;
E3) an expression cassette containing the nucleic acid molecules of E1) and/or E2);
E4) a recombinant vector containing the nucleic acid molecules of E1) and/or E2), or a recombinant vector containing the expression cassette of E3);
E5) recombinant microorganisms containing nucleic acid molecules of E1) and/or E2), or recombinant microorganisms containing expression cassettes of E3), or recombinant microorganisms containing recombinant vectors of E4);
E6) recombinant host cells containing nucleic acid molecules of E1) and/or E2), or recombinant host cells containing expression cassettes of E3), or recombinant host cells containing recombinant vectors of E4).

In the above uses, the nucleic acid molecule of E1) can be any one of the followings:
F1) A DNA molecule having a coding sequence of SEQ ID NO: 2;
F2) A DNA molecule having a nucleotide sequence of SEQ ID NO: 2;
F3) A DNA molecule having a nucleotide sequence of SEQ ID NO: 1.

The DNA molecule shown in SEQ ID NO: 2 can be a coding sequence (CDS) of the ZmLHT1 gene.

An expression of the ZmLHT1 gene can be induced by pathogens of maize southern leaf blight such as Bipolaris maydis, and the ZmLHT1 gene can be a resistance related gene for maize southern leaf blight.

An amino acid sequence encoded by the DNA molecule shown in SEQ ID NO: 2 is the protein ZmLHT1 of SEQ ID NO: 3.

The DNA molecule shown in SEQ ID NO: 1 can be genomic nucleotide sequence of the ZmLHT1 gene.

E1) The nucleic acid molecule may also include a nucleic acid molecule obtained by engineering with codon preference based on the nucleotide sequence shown in SEQ ID NO: 2.

E1) The nucleic acid molecule further includes nucleic acid molecules having 95% or more consistency with a nucleotide sequence as shown in SEQ ID NO: 2 or SEQ ID NO: 1 and derived from the same species and genus.

A coding sequence (CDS) of a gene of a protein ZmLHT1 described in the present invention can be any nucleotide sequence capable of encoding the protein ZmLHT1. Considering the degeneracy of codons and the preference of codons for different species, technicians in the art can use codons suitable for specific species expression as needed.

Further, E2) can be a nucleic acid molecule that reduces expression level of the coding gene of the protein ZmLHT1.

E2) The nucleic acid molecule may be sgRNA, microRNA, siRNA, shRNA, and/or antisense oligonucleotides.

Further, the sgRNA, microRNA, siRNA, shRNA, and/or antisense oligonucleotides are used to inhibit expression of the ZmLHT1 gene.

Furthermore, the nucleic acid molecule of E2) can be sgRNA.

Furthermore, target sequences of the sgRNA can be SEQ ID NO: 14 and SEQ ID NO: 15.

Furthermore, the sgRNA is used to knock out ZmLHT1 gene.

As is well known to those skilled in the art, in addition to inhibition of expression of the ZmLHT1 gene by using gene editing techniques, gene knockdown techniques can also be used to inactivate or silence expression of the ZmLHT1 gene at the post transcriptional or translational level. The gene knockdown technology includes but is not limited to RNA interference, Morpholino interference, antisense nucleic acid, ribozymes, or dominant negative inhibitory mutations. In addition, the use of shRNA or siRNA expressed by viruses such as lentiviruses and adeno-associated viruses to suppress expression of the ZmLHT1 gene and silence the ZmLHT1 gene is also well known to those skilled in the art.

The expression cassette described herein includes a promoter, a nucleic acid molecule encoding the protein ZmLHT1, and a terminator. The promoter can be CaMV35S promoter, NOS promoter, or OCS promoter, and the terminator can be NOS terminator or OCS polyA terminator.

The nucleic acid molecule described herein can be DNA, such as cDNA, genomic DNA, or recombinant DNA; the nucleic acid molecule can also be RNA, such as gRNA, mRNA, siRNA, shRNA, sgRNA, miRNA, or antisense RNA.

The vector described herein refers to a vector that can carry exogenous DNA or target genes into host cells for amplification and expression. The vector can be a cloning vector or an expression vector, including but not limited to plasmids, bacteriophages (such as lambda bacteriophages or M13 filamentous bacteriophages), cosmids (i.e. Cosmids), Ti plasmids, viral vectors (such as retroviruses (including lentiviruses), adenovirus, adeno associated virus, etc.). In one or more embodiments of the present invention, the vector is the vector pCBC-MT1T2, pBUE411, pDR196, pCAMBIASuper1300 mCherry, and/or pEZS-NL.

The microorganisms described herein can be bacteria, fungi, actinomycetes, protozoa, algae, or viruses. Among them, the bacteria may come from the genera Escherichia sp., Erwinia sp., Agrobacterium sp., Flavobacterium sp., Alcaligenes sp., Pseudomonas sp., Bacillus sp., etc., but are not limited thereto. For example, the bacteria may be Escherichia coli, Bacillus subtilis, or Bacillus pumilus. The fungus may be yeast, and the yeast can come from yeast genus (such as Saccharomyces cerevisiae), Kluiveromyces genus (such as Kluyveromyces lactis), Pichia pastoris (such as genus Pichia pastoris), Schizosaccharomyces genus (such as Schizosaccharomyces pombe), Hansenula yeast genus (such as polymorphic Hansenula polymorpha), etc., but not limited thereto. The fungi may also come from the genera Fusarium sp., Rhizoctonia sp., Verticillium sp., Penicillium sp., Aspergillus sp., Cephalosporium sp., etc., but are not limited thereto. The actinomycetes can come from the genera Streptomyces sp., Nocardia sp., Micromonospora sp., Streptosporangium sp., Actinopanes sp., Thermoactinomyces sp., etc., but are not limited thereto. The algae may come from the genera Fucus sp., Achnanthes sp., Amphiphora sp., Amphora sp., Ankistodemus sp., Asteromonas sp., Boekelovia sp., etc., but are not limited thereto. The virus may be rotavirus, herpesvirus, influenza virus, adenovirus, etc., but is not limited thereto. In one or more embodiments of the present invention, the microorganism is Agrobacterium tumefaciens EHA105, yeast mutant 22410a, yeast strain 23344c, and/or Agrobacterium tumefaciens GV3101.

The host cells (also known as recipient cells) described herein can be plant cells or animal cells. The host cell can be understood as referring not only to specific recipient cells, but also to offspring of such cells, and due to natural, accidental, or intentional mutations and/or changes, the offspring may not necessarily be identical to the original parent cells, but still fall within the scope of the host cell. Suitable host cells are known in the art, among them, the plant cells may be Arabidopsis (Arabidopsis thaliana), tobacco (Nicotiana tabacum), maize (Zea mays), rice (Oryza sativa), and wheat (Triticum aestivum), but not limited thereto; the animal cells can be mammalian cells (such as Chinese hamster ovary cells (CHO cells), African green monkey kidney cells (Vero cells), young hamster kidney cells (BHK cells), mouse breast cancer cells (C127 cells), human embryonic kidney cells (HEK293 cells), human HeLa cells, fibroblasts, bone marrow cell lines, T cells or NK cells, etc.), avian cells (such as chicken or duck cells), amphibious cells (such as Xenopus laevis cells or Andrias davidianus cells), fish cells (such as grass carp, carp, rainbow trout or catfish cells), insect cells (such as Sf21 cells or Sf-9 cells), etc, but not limited thereto.

The recombinant vector described herein refers to a recombinant DNA molecule constructed by ligating an exogenous target gene with a vector in vitro.

The recombinant microorganism (or recombinant host cell) described herein refers to a functionally altered recombinant microorganism (or functionally altered recombinant host cell) obtained by manipulation and modification of gene(s) of a target microorganism (or a target host cell). For example, recombinant microorganisms (or recombinant host cells) obtained by introducing exogenous target gene(s) or recombinant vector(s) into target microorganisms (or target host cells), or recombinant microorganisms (or recombinant host cells) obtained by directly genetically editing endogenous gene(s) of target microorganisms (or target host cells). The recombinant microorganism (or recombinant host cell) can be understood not only as a specific recombinant microorganism (or recombinant host cell), but also as offspring of such cells, and due to natural, accidental, or intentional mutations and/or changes, the offspring may not necessarily be identical to original parent cell, but still fall within the scope of recombinant microorganisms (or recombinant host cells).

E4) The recombinant vector can be a recombinant vector pBUE411-ZmLHT1, a recombinant vector pDR196-ZmLHT1, a recombinant vector pCAMBIASuper1300-mCherry ZmLHT1, and/or a recombinant vector pEZS-ZmLHT1.

The recombinant vector pBUE411-ZmLHT1 contains two editing targets (SEQ ID NO: 14 and SEQ ID NO: 15) and a coding gene for Cas9 protein. After being introduced into a recipient, two transcribed guide RNAs can target a target sequence near PAM of a recipient genome through base complementary pairing, that is, targeting ZmLHT1 gene. The Cas9 protein breaks a DNA double strand upstream and downstream of the ZmLHT1 gene, and through organism's own DNA damage repair response mechanism, connects sequences at both ends of the breaked upstream and downstream, thereby achieving knockout of the ZmLHT1 gene.

A preparation method of the recombinant vector pBUE411-ZmLHT1 includes the following steps: using an intermediate vector pCBC-MTIT2 as a template, performing four primer PCR amplification by using primers Spacer 1-F (SEQ ID NO: 4), Spacer 1-R (SEQ ID NO: 5), Spacer 2-F (SEQ ID NO: 6), and Spacer 2-R (SEQ ID NO: 7), and cloning resulting PCR product (containing dual targets of SEQ ID NO: 14 and SEQ ID NO: 15) into a vector pBUE411 to obtain a ZmLHT1 gene editing vector, namely the recombinant vector pBUE411-ZmLHT1, for gene knockout.

The recombinant vector pDR196-ZmLHT1 is a recombinant expression vector obtained by replacing a fragment (a small fragment) between EcoRI and XhoI recognition sites of a pDR196 vector with a DNA fragment having a nucleotide sequence of SEQ ID NO: 2 in the sequence listing, while remaining other nucleotide sequences of the pDR196 vector unchanged. After introducing the recombinant vector pDR196-ZmLHT1 into a host, ZmLHT1 protein having an amino acid sequence as shown in SEQ ID NO: 3 is expressed.

The recombinant vector pCAMBIASuper1300-mCherry-ZmLHT1 is a recombinant expression vector obtained by replacing a fragment (a small fragment) between XmaI and KpnI recognition sites of a pCAMBIASuper1300-mCherry vector with a DNA fragment having a nucleotide sequence of SEQ ID NO: 2 in the sequence listing, while remaining other nucleotide sequences of the pCAMBIASuper1300-mCherry vector unchanged.

The recombinant vector pEZS-ZmLHT1 is obtained by replacing a fragment (a small fragment) between HindIII and BamHI recognition sites of a pEZS-NL vector with a DNA fragment having a nucleotide sequence of SEQ ID NO: 2 in the sequence listing, while remaining other nucleotide sequences of the pEZS-NL vector unchanged.

E5) The recombinant microorganism can be a recombinant Agrobacterium tumefaciens EHA105/pBUE411-ZmLHT1, a recombinant strain 22 Δ 10a/pDR196, a recombinant strain 22 Δ 10a/pDR196-ZmLHT1, and/or a recombinant strain 23344c/pDR196.

The EHA105/pBUE411-ZmLHT1 is a recombinant microorganism obtained by introducing the recombinant vector pBUE411-ZmLHT1 into Agrobacterium tumefaciens EHA105.

The 22 Δ 10a/pDR196-ZmLHT1 is a recombinant microorganism obtained by introducing the recombinant vector pDR196-ZmLHT1 into yeast mutant 22 Δ 10a.

The 23344c/pDR196 is a recombinant microorganism obtained by introducing the recombinant vector pDR196 into yeast strain 23344c.

The 22 Δ 10a/pDR196 is a recombinant microorganism obtained by introducing the recombinant vector pDR196 into yeast mutant 22 Δ 10a.

The recombinant microorganism can also be a recombinant microorganism obtained by introducing the recombinant vector pCAMBIASuper1300-mCherry-ZmLHT1 or the recombinant vector pEZS-ZmLHT1 into Agrobacterium tumefaciens GV3101.

The introducing can be an introducing achieved through recombinant methods, including but not limited to Agrobacterium mediated transformation, biologistic methods, electroporation, in plant technology, freeze-thaw methods, and so on.

The present invention also provides a method for cultivating disease resistant plants, the method may include reducing content and/or activity of the protein ZmLHT1 in target plants to obtain disease resistant plants with higher disease resistance than the target plants.

In the above methods, reducing content and/or activity of the protein ZmLHT1 in target plants can be achieved by reducing expression level and/or activity of a coding gene of the protein ZmLHT1 in the target plants.

In the above methods, reducing expression level and/or activity of a coding gene of the protein ZmLHT1 in the target plant can be achieved by reducing or inactivating activity of a coding gene of the protein ZmLHT1 in a genome of the target plant using gene mutation, gene knockout, gene editing, or gene knockdown techniques.

In the above methods, reducing or inactivating activity of a coding gene of the protein ZmLHT1 in a genome of the target plant using gene editing technique can be performed using a CRISPR/Cas9 system. The CRISPR/Cas9 system includes a vector expressing a sgRNA targeting a coding gene of the protein, and a target sequence of the sgRNA can be SEQ ID NO: 14 and SEQ ID NO: 15.

The method for cultivating disease resistant plants according to the present invention may include the following steps: inhibiting an expression of a nucleic acid molecule capable of expressing ZmLHT1 protein in a target plant to obtain a transgenic plant; the transgenic plant has improved disease resistance compared to the target plant. Among them, inhibiting an expression of a nucleic acid molecule capable of expressing ZmLHT1 protein in a target plant can be achieved through any technical means that can achieve this objective, such as specific cleavage of the nucleic acid molecule by sequence specific nucleases (such as CRISPR/Cas9 nucleases), thereby reducing its expression in the target plant. The method of cultivating disease resistant plants can be achieved through crossing or transgenic methods.

In one embodiment of the present invention, the method for cultivating disease resistant plants includes the following steps:
(1) constructing a CRISPR/Cas9 gene editing vector targeting SEQ ID NO: 14 and SEQ ID NO: 15;
(2) introducing the CRISPR/Cas9 gene editing vector constructed in step (1) into a target plant;
(3) obtaining disease resistant plants with higher disease resistance than the target plant through screening and identification.

In the above method, the CRISPR/Cas9 gene editing vector can be the recombinant vector pBUE411-ZmLHT1.

In the above method, the target plant can be maize, specifically maize inbred line B73-329.

In the above method, the introducing includes but is not limited to: transfecting plant cells or tissues by using conventional biological methods such as Ti plasmid, Ri plasmid, plant virus vector, direct DNA transformation, microinjection, electroporation, Agrobacterium mediated method, etc., and cultivating the transfected plant cells or tissues into plants.

In the above methods, the introducing can specifically be Agrobacterium mediated method.

The present invention also provides a method for preparing a maize with improved disease resistance, the method may include the following steps: mutating ZmLHT1 gene shown in SEQ ID NO: 1 in the sequence listing in a maize genome into ZmLHT1/+2 bp gene, ZmLHT1/−8 bp gene, or ZmLHT1/−25 bp gene, to obtain a maize with improved disease resistance; a nucleotide sequence of the ZmLHT1/+2 bp gene is as shown in SEQ ID NO: 16, a nucleotide sequence of the ZmLHT1/−8 bp gene is as shown in SEQ ID NO: 17, and a nucleotide sequence of the ZmLHT1/−25 bp gene is as shown in SEQ ID NO: 18.

The present invention also provides use of the ZmLHT1/+2 bp gene, ZmLHT1/−8 bp gene, or ZmLHT1/−25 bp gene, or the ZmLHT1/+2 bp gene, ZmLHT1/−8 bp gene, or ZmLHT1/−25 bp gene in improving maize disease resistance.

A nucleotide sequence of the ZmLHT1 gene mutant ZmLHT1/+2 bp gene is as shown in SEQ ID NO: 16, which involves inserting two bases “A and T” into the first exon of ZmLHT1 gene (corresponding to positions 40 and 65 of SEQ ID NO: 2).

A nucleotide sequence of the ZmLHT1 gene mutant ZmLHT1/−8 bp is as shown in SEQ ID NO: 17, which involves a total of 8 bases “GCA and TATTC” deletion in the first exon of ZmLHT1 gene (corresponding to positions 39-41 and 62-66 of SEQ ID NO: 2).

A nucleotide sequence of the ZmLHT1 gene mutant ZmLHT1/−25 bp is as shown in SEQ ID NO: 18, which involves a deletion of 25 bases “AAGGGCCGCCAGCCGCCAGCTATTC” (SEQ ID NO: 19, corresponding to positions 41-65 of SEQ ID NO: 2) in the first exon of ZmLHT1 gene.

The present invention also provides a disease resistant plant obtained by any one of the methods for cultivating disease resistant plants described herein.

Herein, the plant can be any one of the followings:
G1) monocotyledonous or dicotyledonous plants;
G2) poaceae plants;
G3) plants of genus Zea.

Furthermore, the disease resistance may be resistance to southern leaf blight.

The present invention further provides maize with improved disease resistance prepared by any one of methods described herein.

Further, the disease resistance may be resistance to southern leaf blight.

Herein, the plant can be crops (such as a farm crop).

The plants of genus Zea can be maize, such as maize inbred line B73-329 and/or maize inbred line B73.

The present invention also provides use of the method for cultivating disease resistant plants in creation of disease resistant plants and/or plant breeding.

The ZmLHT1 gene described herein can be a resistance related gene for maize southern leaf blight.

The plant breeding described herein can be crop disease resistance breeding, specifically maize resistance to southern leaf blight breeding. The purpose of the breeding is to select and cultivate maize with improved resistance to southern leaf blight.

The plant breeding described herein can be molecular breeding that utilizes the ZmLHT1 gene and/or protein ZmLHT1 described in the present invention to increase crop disease resistance.

The disease resistant described herein may be resistant to southern leaf blight, and the disease resistance may be resistance to southern leaf blight.

The regulating plant disease resistance described herein can be increasing (upregulating) plant disease resistance or decreasing (downregulating) plant disease resistance.

Specifically, the regulating plant disease resistance described herein can be regulating plant disease resistance to southern leaf blight, including increasing (upregulating) plant resistance to southern leaf blight or decreasing (downregulating) plant resistance to southern leaf blight.

The southern leaf blight described herein can be a southern leaf blight caused by maize Bipolaris maydis.

Herein, the disease resistant plants are understood to include not only the first generation transgenic plants obtained by knocking out the ZmLHT1 gene, but also their offsprings. The disease resistant plants include seeds, callus tissue, intact plants, and cells.

The present invention discloses use of a maize amino acid transporter gene ZmLHT1 in disease resistant genetic engineering. The maize amino acid transporter gene ZmLHT1 is involved in regulating maize resistance to southern leaf blight, and its expression is induced by pathogen of maize southern leaf blight.

The present invention obtained maizes (ZmLHT1 mutants: ZmLHT1^{2 bp}, ZmLHT1^{8 bp}, and ZmLHT1^{25 bp} lines) by knocking out a maize ZmLHT1 gene based on the CRISPR-Cas9 system. Further identification for disease resistance of ZmLHT1 mutants was conducted and its field disease grades were evaluated. Experimental results have shown that after inoculation with Race O of Bipolaris maydis, compared with wild-type maize without ZmLHT1 gene knockout, maize with ZmLHT1 gene knockout (ZmLHT1 mutant) has a significantly higher disease attack level than that of wild-type maize, demonstrating excellent resistance to southern leaf blight. The resistance of maize plants to southern leaf blight is significantly enhanced after ZmLHT1 gene has been knockout. In addition, under normal environment, mutation of the ZmLHT1 gene did not cause loss in yield, while in an environment of southern leaf blight, mutation of the ZmLHT1 gene significantly increased maize yield. DAB staining analysis revealed that leaves of ZmLHT1 gene knockout maize (ZmLHT1 mutant) accumulated a large amount of reactive oxygen species (ROS), indicating that the ZmLHT1 gene is mainly involved in clearing ROS and causing maize susceptibility. It can be seen that knocking out ZmLHT1 gene through CRISPR/Cas9 technology significantly enhances maize resistance to southern leaf blight, and the ZmLHT1 gene can be applied to disease resistant breeding of maize southern leaf blight.

ZmLHT1 gene and its encoded ZmLHT1 protein identified in the present invention can regulate plant disease resistance (such as resistance to southern leaf blight). By reducing content and/or activity of ZmLHT1 protein in a target plant (such as knocking out ZmLHT1 gene), the disease resistance of the target plant can be significantly improved. By increasing content and/or activity of ZmLHT1 protein in a target plant (such as overexpression of ZmLHT1 gene), the disease resistance of the target plant can be significantly reduced. A nucleotide sequence of ZmLHT1 genome is as shown in SEQ ID NO: 1, with a full length of 2803 bp. A nucleotide sequence of a coding sequence (CDS) of the ZmLHT1 gene is as shown in SEQ ID NO: 2, with a length of 1419 bp and encoding 472 amino acids. The encoded amino acid sequence is as shown in SEQ ID NO: 3. A protein encoded by ZmLHT1 gene is named ZmLHT1 protein (SEQ ID NO: 3).

The present invention discovered for the first time a use of ZmLHT1 gene and its encoded protein in improving disease resistance of maize, which is of great significance and broad application prospects for cultivating new varieties of maize resistant to southern leaf blight, overcoming the shortcomings of traditional breeding, promoting the commercialization of maize breeding process, and ensuring high and stable yield of maize.

### BRIEF DESCRIPTION OF THE DRAWINGS

FIG. 1 shows identification of ZmLHT1 mutant plant line.

FIGS. 2A-2F show resistance to southern leaf blight and yield analysis of ZmLHT1 mutant plant line and wild-type maize. FIGS. 2A-2B show analysis of resistance to southern leaf blight of ZmLHT1 mutant plant line and wild-type maize; FIGS. 2C-2D show yield analysis of ZmLHT1 mutant plant line and wild-type maize under normal environments (an environment without southern leaf blight); FIGS. 2E-2F show yield analysis of mutant plant line and wild-type maize under environments of southern leaf blight.

FIG. 3 shows identification of amino acid transportation function of ZmLHT1 in yeast mutants.

FIG. 4 shows fluorescence quantitative analysis of ZmLHT1 gene after inoculation with Bipolaris maydis.

FIG. 5 shows identification of subcellular localization of a protein encoded by ZmLHT1 gene.

FIG. 6 shows DAB staining analysis of ZmLHT1 mutant plant line and wild-type maize after inoculation with Bipolaris maydis. The zmlht1^{8 bp} in FIG. 6 represents ZmLHT1 mutant plant line ZmLHT^{18 bp}.

### DETAILED DESCRIPTION OF THE EMBODIMENTS

The present invention is further described in detail in conjunction with specific embodiments below. The provided examples are only intended to illustrate the present invention and not to limit its scope. The examples provided below can serve as a guide for those skilled in the art to make further improvement and do not in any way limit the present invention.

The experimental methods in the following examples, unless otherwise specified, are all conventional methods, carried out according to the techniques or conditions described in the literature in the art or according to the product instructions. The materials, reagents, etc. used in the following examples can be obtained from commercial sources unless otherwise specified.

The vectors pCBC-MT1T2 and pBUE411 in the following examples are described in the literature: Xing H L, et al. A CRISPR/Cas9 toolkit for multiplex genome editing in plants. BMC plant biology, 2014, 14(1): 1-12.

The following examples use GraphPad statistical software to process data, and the experimental results are expressed as mean±standard deviation, using t-test method. P<0.05 (*) indicates statistical difference, P<0.01 (**) indicates significant statistical difference, and P<0.001 (***) indicates extremely significant statistical difference.

The quantitative experiments in the following examples, unless otherwise specified, are set in triplicate and the results are taken as mean.

### Example 1: Sequence of Maize ZmLHT1 Gene

Obtaince of maize ZmLHT1 gene: after extensive and intensive research, the inventors of the present application used maize genome database website maizeGDB to analyze the maize genome sequence and found a gene Zm00001d035162, named as ZmLHT1. A nucleotide sequence of the ZmLHT1 genome is as shown in SEQ ID NO: 1, with a full length of 2803 bp. A nucleotide sequence of a coding sequence (CDS) of the ZmLHT1 gene is as shown in SEQ ID NO: 2, with a length of 1419 bp and encoding 472 amino acids. The encoded amino acid sequence is as shown in SEQ ID NO: 3. A protein encoded by the ZmLHT1 gene is named ZmLHT1 protein (SEQ ID NO: 3).

### Example 2: Creation and Identification of ZmLHT1 Mutant Plants

### 1. Creation of ZmLHT1 Mutant Plants

ZmLHT1 mutant materials were created using CRISPR/Cas9 technology. Firstly, based on ZmLHT1 gene sequence, target sequences containing NGG or CCN structures were designed using a CRISPR/Cas9 system.

Target 1:
(SEQ ID NO: 14)
5′-TGGAGATGACGACGCAGCAAGGG-3′,
Target 2:
(SEQ ID NO: 15)
5′-GCCAGCCGCCAGCTATTCTCCGG-3′.
Target 1 and target 2 were constructed into a pBUE411 vector, and primers were
designed as follows:
Spacer 1-F:
(SEQ ID NO: 4)
5′-AATAATGGTCTCAGGCGGGAGATGACGACGCAGCAA,
Spacer 1-R:
(SEQ ID NO: 5)
5′-ATTATTGGTCTCTAAACGAGAATAGCTGGCGGCTGG,
Spacer 2-F:
(SEQ ID NO: 6)
5′-gGGAGATGACGACGCAGCAAgttttagagctagaaatagca,
Spacer 2-R:
(SEQ ID NO: 7)
5′-GAGAATAGCTGGCGGCTGGcgcttcttggtgccgc.

Four-primer PCR amplification was performed using pCBC-MT1T2 as a template, and PCR product (containing dual targets) was purified and recovered. A Golden-gate enzyme clearvage-ligation system was established, and the PCR product was transferred into a final expression plasmid pBUE411 containing Cas9 to obtain a ZmLHT1 gene editing vector (i.e. a recombinant vector for gene knockout), named pBUE411-ZmLHT1.

The recombinant vector pBUE411-ZmLHT1 contains two editing targets (SEQ ID NO: 14 and SEQ ID NO: 15) and a coding gene for the Cas9 protein. After being introduced into a recipient, the two transcribed guide RNAs can target target sequences near PAM of a recipient genome through base complementary pairing, that is, targeting ZmLHT1 gene. The Cas9 protein breaks a DNA double strand upstream and downstream of the ZmLHT1 gene, and through organism's own DNA damage repair response mechanism, connects sequences at both ends of the breaked upstream and downstream, thereby achieving knockout of the ZmLHT1 gene.

The recombinant vector pBUE411-ZmLHT1 sequenced to be correct was transformed into Agrobacterium tumefaciens EHA105 using freeze-thaw method, obtaining recombinant Agrobacterium tumefaciens EHA105/pBUE411-ZmLHT1.

ZmLHT1 CRISPR/Cas9 mutant plants (i.e. TO generation gene edited plants) were obtained by infecting immature embryos of maize inbred line B73-329 with Agrobacterium tumefaciens (EHA105/pBUE411 ZmLHT1).

### 2. Identification of ZmLHT1 Mutant Plants

Upstream primer 5′-TTAACAAACCAAGCTGAGATGC-3′ (SEQ ID NO: 8) and downstream primer 5′-CAGCACGAAGTGGCAGGA-3′ (SEQ ID NO: 9) were designed near the ZmLHT1 gene editing site. Leaves of transgenic TO generation maize seedlings were collected and extracted for genomic DNA. Using the genomic DNA as a template for PCR amplification, the amplified products were sequenced and then compared with wild-type sequences to identify effective mutant plant lines, which were named ZmLHT1^{2 bp}, ZmLHT1^{8 bp}, and ZmLHT1^{25 bp}, respectively. Among them:

ZmLHT1^{2 bp} involves insertion of two bases “A” and “T” (corresponding to positions 40 and 65 of SEQ ID NO: 2) into the first exon of ZmLHT1 gene, resulting in premature termination of coding and knocking out the ZmLHT1 gene, correspondingly a mutant ZmLHT1/+2 bp of the ZmLHT1 gene was obtained. A nucleotide sequence of the mutant ZmLHT1/+2 bp gene of the ZmLHT1 gene is as shown in SEQ ID NO: 16.

ZmLHT1^{8 bp} involves deletion of a total of 8 bases “GCA and TATTC” (corresponding to positions 39-41 and 62-66 of SEQ ID NO: 2) in the first exon of the ZmLHT1 gene, resulting in premature termination of coding. As a result, the ZmLHT1 gene was knocked out, and correspondingly a mutant ZmLHT1/−8 bp of the ZmLHT1 gene was obtained. A nucleotide sequence of the mutant ZmLHT1/−8 bp of the ZmLHT1 gene is as shown in SEQ ID NO: 17.

ZmLHT1^{25 bp} involves deletion of 25 bases “AAGGGCCGCCAGCCGCCAGCTATTC” (SEQ ID NO: 19, corresponding to positions 41-65 of SEQ ID NO: 2) in the first exon of the ZmLHT1 gene resulted in premature termination of coding. As a result, the ZmLHT1 gene was knockout and correspondingly a mutant ZmLHT1/−25 bp of the ZmLHT1 gene was obtained. A nucleotide sequence of the mutant ZmLHT1/−25 bp gene of the ZmLHT1 gene is as shown in SEQ ID NO: 18.

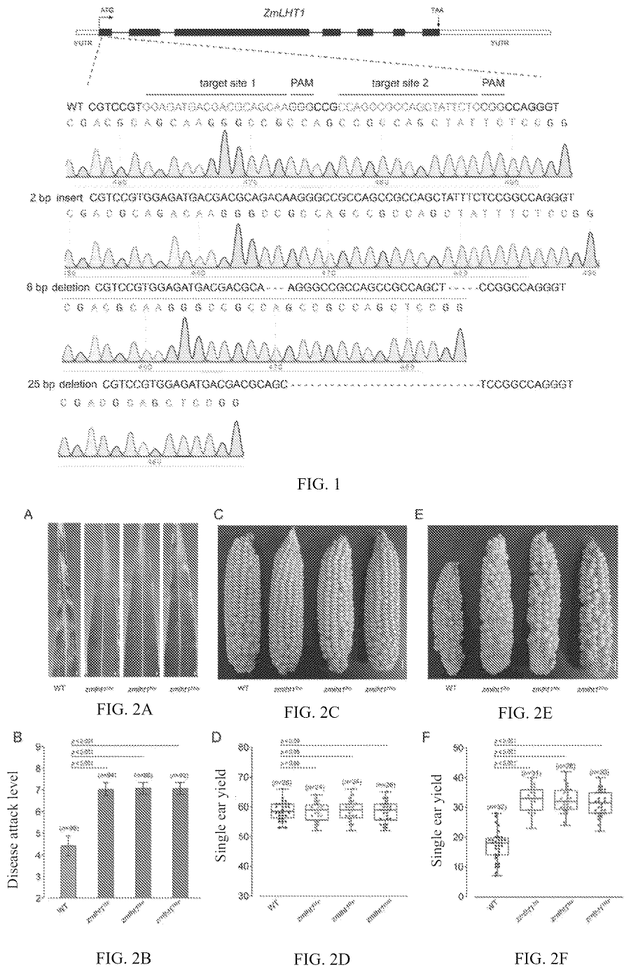

The identification results of the ZmLHT1 mutant plant lines were shown in FIG. 1. The sequences in FIG. 1 were all near the targets of gene editing materials. Among them, WT in FIG. 1 represents a sequence of wild-type gene fragment (SEQ ID NO: 20), 2 bp insert represents an insertion type fragment with two bases inserted on the basis of wild-type gene fragment (SEQ ID NO: 21), 8 bp deletion represents deletion type fragment with 8 bases deleted on the basis of wild-type gene fragment (SEQ ID NO: 22), and 25 bp deletion represents deletion type fragment with 25 bases deleted on the basis of wild-type gene fragment (SEQ ID NO: 23). A nucleotide sequence of sequenced fragment (the next line of WT) of the wild-type portion in FIG. 1 is SEQ ID NO: 24, a nucleotide sequence of sequenced fragment (the next line of 2 bp insert) of the 2 bp insertion type portion is SEQ ID NO: 25, a nucleotide sequence of sequenced fragment (the next line of 8 bp deletion) of the 8 bp deletion type portion is SEQ ID NO: 26, and a nucleotide sequence of sequenced fragment (the next line of 25 bp deletion) of the 25 bp deletion type portion is SEQ ID NO: 27.

### Example 3: Field Phenotype Validation of ZmLHT1 Mutant Plants

The tested plants were:
1. Wild type maize: maize inbred line B73-329;
2. ZmLHT1 mutant plants: T3 generation plants of ZmLHT12 bp line, T3 generation plants of ZmLHT18 bp line, T3 generation plants of ZmLHT125 bp lines (at least 100 plants per line).

### Field Assessment of Disease Attack Level of ZmLHT1 Mutant

Wild type maize and ZmLHT1 mutant plants were planted in the transgenic experimental base of Caoxinzhuang Village Agricultural Experimental Station in Yangling Town, Yangling District, Xianyang City, Shanxi Province (34° 18′27″N, 108° 5′58″ E). The Race O of Bipolaris maydis, which had been isolated in the laboratory, was propagated using PDA medium. In a light incubator, it was then cultured under light for 12 hours and under dark conditions for 12 hours (temperature 25° C.). After mycelium developed well, conidia were eluted off the PDA medium to make spore suspension. After being soaked for two days, sorghum grains were loaded into 1000 ml a sterilize conical flask, then spore suspension was added and mixed well at 26° C. for cultivation. The flask was shaken vigorously once a day to avoid clumping. After being covered with mycelium, the sorghum grains were dried in the dark for later use. At 6-leaf stage of maize, sorghum grains infected with mycelium were injected into maize horn mouth (about 25 sorghum grains inoculated per plant). Phenotype investigations were conducted one week before and one week after maize silking (incidence of disease in maize leaves were investigated). The reference for disease attack level is as follows: Sermons S M, et al. Large scale field inoculation and scoring of maize southern leaf blight and other maize foliar fungal diseases. Bio-protocol, 2018, 8(5): e2745. The results showed that ZmLHT1 mutant had a significantly higher disease attack level than that of the wild type (FIGS. 2A-2B), demonstrated excellent resistance to southern leaf blight, indicating that maize plants with knockout ZmLHT1 gene significantly enhanced the disease resistance.

Yield Analysis of ZmLHT1 Mutant with and without Inoculation

Wild type maize and ZmLHT1 mutant plants were planted in a transgenic experimental base of Caoxinzhuang Village, Yangling Town, Yangling District, Xianyang City, Shaanxi Province (34° 18′27 ″N, 108° 5′58″ E). They were planted on flat ground without ridges, with a row length of 3 meters, 13 plants per row, a row spacing of 0.6 meters, and a plant spacing of 0.24 meters. Yield analysis was conducted on ears after maturity harvested under inoculated (FIGS. 2E-2F) and non-inoculated environments (FIGS. 2C-2D). The results showed that under a normal environment (without inoculation), mutation of the ZmLHT1 gene did not cause any loss in yield, while under an environment of southern leaf blight, mutation of the ZmLHT1 gene significantly increased maize yield.

### Example 4: Identification of Amino Acid Transport Function of ZmLHT1 Gene in Yeast Mutants

The tested plant was: maize inbred line B73

In order to determine the amino acid transport function of a protein ZmLHT1 encoded by a ZmLHT1 gene, functional complementation experiments were conducted in yeast mutants. The steps were as follows:
1. Extracting total RNA from B73 leaves and reverse transcribing it to obtain cDNA.
2. Using the cDNA obtained in step 1 as a template, PCR amplification was performed using a primer pair consisting of primer5′-CGCTCTCCAATCTCCCATT-3′ (SEQ ID NO: 10) and primer 5′-ACGGCAAAGAACTCGCACT-3′ (SEQ ID NO: 11) to obtain a PCR amplification product (ZmLHT1 gene).
3. Inserting the ZmLHT1 gene into a yeast expression vector pDR196 to obtain a recombinant vector pDR196-ZmLHT1.

The recombinant vector pDR196-ZmLHT1 is a recombinant expression vector obtained by replacing a fragment (small fragment) between EcoRI and XhoI recognition sites of pDR196 vector with a DNA fragment of SEQ ID NO: 2 in the sequence listing, while remaining other nucleotide sequences of the pDR196 vector unchanged. After being introduced into a host, the recombinant vector pDR196-ZmLHT1 expressed a ZmLHT1 protein with the amino acid sequence as shown in SEQ ID NO: 3.
4. Transforming the recombinant vectors pDR196-ZmLHT1 and pDR196 into yeast mutant 22 Δ 10a by LiAc/ss carrier DNA/PEG method, to obtain recombinant strains 22 Δ 10a/pDR196-ZmLHT1 and 22 Δ 10a/pDR196. The recombinant strain 23344c/pDR196 obtained by transforming the recombinant vector pDR196 into yeast strain 23344c was used as a positive control yeast strain, and 22 Δ 10a/pDR196 was used as a negative control yeast strain.
5. Culturing the transformed recombinant strain 22 Δ 10a/pDR196-ZmLHT1, 22 Δ 10a/pDR196, and recombinant strain 23344c/pDR196 in YNB solid and liquid media containing 1 mM Ala, Arg, Gln, Glu, GABA, Phe, Pro, Leu, or ammonium sulfate (at a temperature of 30° C.).

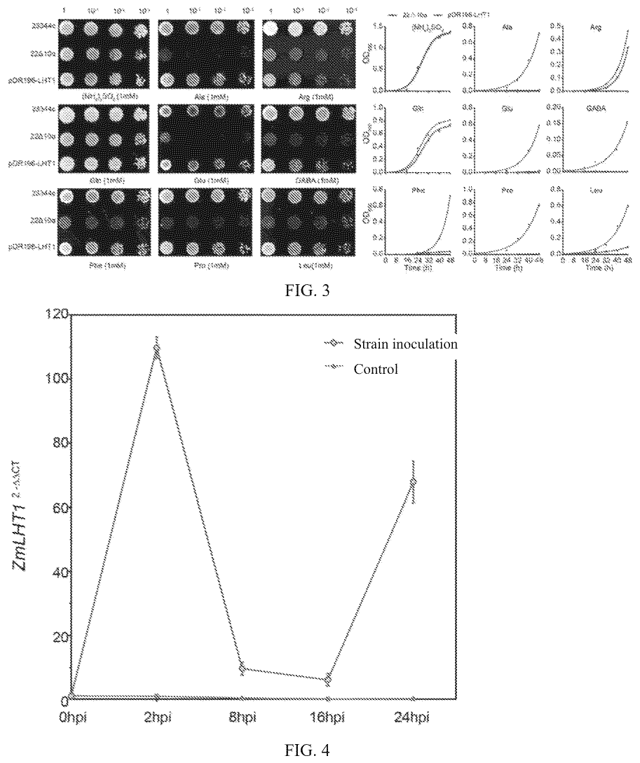

The results showed that when cultured in solid and liquid media containing different amino acids, the growth rate of positive strain overexpressing the ZmLHT1 gene (recombinant strain 22 Δ 10a/pDR196-ZmLHT1) was significantly different from that of an empty vector control (recombinant strain 22 Δ 10a/pDR196) (FIG. 3), indicating that ZmLHT1 has amino acid transport function.

### Example 5: Fluorescence Quantitative Analysis of ZmLHT1 Gene after Inoculation with Bipolaris maydis

The tested plant was maize inbred line B73.

The maize inbred line B73 was cultured in a greenhouse for 28 days, and Race O of Bipolaris maydis was propagated with PDA medium. The cultured spores were eluted off the medium to make a spore suspension (spore liquid concentration 5×10⁴/ml). When the maize grew to four leaf stage, the spore suspension was evenly sprayed onto both sides of maize leaves (the fourth leaf) using an air pump for inoculation. Subsequently, RNA was extracted from the leaves and reverse transcribed to synthesize the first strand of cDNA. According to qRT-PCR primer design requirements, Primer Premier 5.0 software was used to design fluorescent quantitative primers for ZmLHT1 gene, qRT-LHT1-F: 5′-TACTGGGCTTTCGGCGATA-3′ (SEQ ID 5′-NO: 12), qRT-LHT1-R: TGAACATTGTGAACGCAACG-3′ (SEQ ID NO: 13). Expression of ZmLHT1 gene in the leaves of maize after inoculation with Bipolaris maydis was detected using a fluorescence quantitative analyzer. The results showed that the expression level of ZmLHT1 gene was rapidly upregulated after inoculation with Race O of Bipolaris maydis, with the highest expression level at two hours, followed by a decrease, and then upregulated again at 16 hours after inoculation (FIG. 4). This indicated that the expression of ZmLHT1 gene is induced by maize Bipolaris maydis, and the ZmLHT1 gene is a resistance related gene for maize southern leaf blight.

### Example 6: Identification of Subcellular Localization of ZmLHT1 Gene Encoded Protein

The tobacco used in this example was Nicotiana benthamiana.

A coding sequence of ZmLHT1 gene (SEQ ID NO: 2) was amplified from cDNA of maize B73 leaf, and the coding sequence of ZmLHT1 gene was inserted into pCAMBIAupper1300-mCherry and pEZS-NL expression vectors, respectively, to obtain recombinant vectors pCAMBIAupper1300-mCherry-ZmLHT1 and pEZS-ZmLHT1.

The recombinant vector pCAMBIASuper1300-mCherry-ZmLHT1 is a recombinant expression vector obtained by replacing a fragment (small fragment) between XmaI and KpnI recognition sites of a pCAMBIASuper1300-mCherry vector with a DNA fragment of SEQ ID NO: 2 in the sequence listing, while remaining other nucleotide sequences of the pCAMBIASuper1300-mCherry vector unchanged.

The recombinant vector pEZS-ZmLHT1 is a recombinant expression vector obtained by replacing a fragment (small fragment) between HindIII and BamHI recognition sites of a pEZS-NL vector with a DNA fragment having a nucleotide sequence of SEQ ID NO: 2 in the sequence listing, while remaining other nucleotide sequences of the pEZS-NL vector unchanged.

The pCAMBIAsuper1300 fusion expression vector (i.e. recombinant vector pCAMBIAsuper1300-mCherry-ZmLHT1) was transformed into Agrobacterium tumefaciens GV3101 by freeze-thaw method. Subsequently, transient expression of ZmLHT1-mCherry fusion protein was performed on tobacco leaves using Agrobacterium mediated methods.

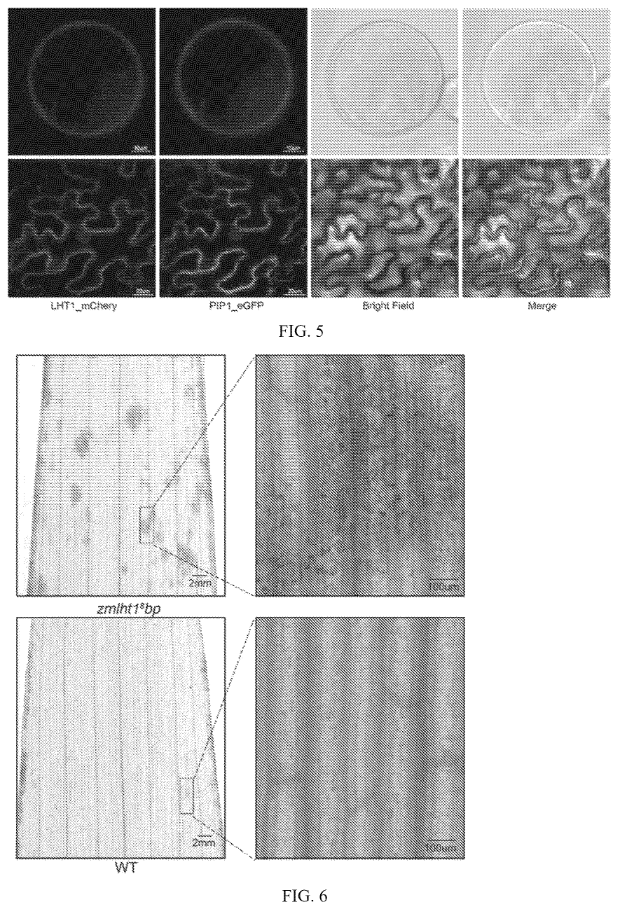

Using polyethylene glycol (PEG)-mediated maize protoplast transformation method, a pEZS fusion expression vector (i.e. recombinant vector pEZS-ZmLHT1) was transformed into maize protoplasts for transient expression of ZmLHT1-mCherry fusion protein. The fluorescence microscope observation results showed that the ZmLHT1 protein was localized on cytoplasmic membrane of tobacco leaves and maize protoplast cells (FIG. 5).

### Example 7: DAB Staining Analysis of ZmLHT1 Mutant Plant Line and Wild-Type after Inoculation with Maize Bipolaris maydis

The tested plants were:
1. Wild type maize: maize inbred line B73-329;
2. ZmLHT1 mutant plants: T3 generation plants of ZmLHT12 bp line, T3 generation plants of ZmLHT18 bp line, T3 generation plants of ZmLHT125 bp line (at least 10 plants per line).

Plants produce a large amount of reactive oxygen species (ROS) in their bodies during pathogen infection to enhance their own immunity. We used DAB staining to detect the changes of ROS content in ZmLHT1 mutant plants and wild-type maize after inoculation with maize Bipolaris maydis. Maize seedlings, after being cultivated in greenhouse for 14 days, were inoculated with Race O of Bipolaris maydis, then leaves of ZmLHT1 mutant plants and wild-type maize were collected for DAB staining (staining method refer to: Daudi A, et al. Detection of hydrogen peroxide by DAB staining in Arabidopsis leaves. Bio-Protocol, 2012, 2 (18): e263. The results showed that the leaves of wild-type maize had almost no accumulation of ROS, while the leaves of ZmLHT1 mutant plants accumulated a large amount of ROS (FIG. 6). This indicated that the ZmLHT1 gene is mainly involved in clearance of ROS, causing maize susceptibility.

In summary, ZmLHT1 gene and its encoded ZmLHT1 protein identified by the present invention can regulate plant disease resistance (such as resistance to southern leaf blight). By reducing content and/or activity of ZmLHT1 protein in a target plant (such as knocking out the ZmLHT1 gene), the disease resistance of the target plant can be significantly improved. By increasing content and/or activity of ZmLHT1 protein in a target plant (such as overexpression of the ZmLHT1 gene), the disease resistance of the target plant can be significantly reduced.

The present invention has been described in detail above. For those skilled in the art, the present invention can be implemented within a wide range under equivalent parameters, concentrations, and conditions without departing from the purpose and scope of the present invention, and without the need for unnecessary experimentation. Although the present invention provides specific embodiments, it should be understood that further improvements can be made to the present invention. In summary, according to the principles of the present invention, this application is intended to include any modifications, uses, or improvements to the present invention, including changes made using conventional techniques known in the art that depart from the scope already disclosed in this application. According to the scope of the attached claims, some basic features can be applied.

### INDUSTRY APPLICATION

ZmLHT1 gene and its encoded ZmLHT1 protein identified in the present invention can regulate plant disease resistance (such as resistance to southern leaf blight). By reducing content and/or activity of ZmLHT1 protein in a target plant (such as knocking out the ZmLHT1 gene), the disease resistance of the target plant can be significantly improved. By increasing content and/or activity of ZmLHT1 protein in a target plant (such as overexpression of the ZmLHT1 gene), the disease resistance of the target plant can be significantly reduced. ZmLHT1 gene can be applied in breeding of southern leaf blight-resistant maize, which is beneficial to promoting the commercialization of maize breeding process.

What is claimed is:

1. A method for using a protein or a substance that regulates an activity and/or a content of the protein for one of the following purposes:
A1) regulating a disease resistance of a plant;
A2) a preparation of products that regulate the disease resistance of the plant;
A3) cultivating disease resistant plants;
A4) a preparation of products for cultivating the disease resistant plants;
A5) plant breeding or an improvement of plant germplasm resources;
the protein is one of the followings:
B1) a protein having the amino acid sequence of SEQ ID NO. 3;
B2) a protein obtained by substituting and/or deleting and/or adding at least one amino acid residue to the amino acid sequence shown in SEQ ID NO: 3, which has 80% or more identity with and a same function as the protein in B1);
B3) a fusion protein obtained by linking at least one tag at an N-terminus and/or a C-terminus of the protein in B1) or B2) and has the same function.

2. The method according to claim 1, wherein the protein is derived from maize.

3. A method for using a biomaterial related to the protein in the method according to claim 1 for one of the following purposes:
D1) the disease resistance of the plant;
D2) the preparation of the products regulating the disease resistance of the plant;
D3) cultivating the disease resistant plants;
D4) the preparation of the products for cultivating the disease resistant plants;
D5) the plant breeding or the improvement of the plant germplasm resources;
the biomaterial is one of the following E1) to E6):
E1) a nucleic acid molecule encoding the protein;
E2) a nucleic acid molecule that inhibits or reduces expression of a coding gene of the protein;
E3) an expression cassette containing the nucleic acid molecules of E1) and/or E2);
E4) a recombinant vector containing the nucleic acid molecules of E1) and/or E2), or a recombinant vector containing the expression cassette of E3);
E5) a recombinant microorganism containing the nucleic acid molecules of E1) and/or E2), or a recombinant microorganism containing the expression cassette of E3), or a recombinant microorganism containing the recombinant vector of E4);
E6) a recombinant host cell containing the nucleic acid molecules of E1) and/or E2), or a recombinant host cell containing the expression cassette of E3), or a recombinant host cells containing the recombinant vector of E4).

4. The method according to claim 3, wherein the nucleic acid molecule of E1) is one of the followings:
F1) a DNA molecule having the coding sequence of SEQ ID NO: 2;
F2) a DNA molecule having the nucleotide sequence of SEQ ID NO: 2;
F3) a DNA molecule having the nucleotide sequence of SEQ ID NO: 1.

5. A method for cultivating a disease resistant plant, comprising reducing the content and/or the activity of the protein in the method according to claim 1 in a target plant to obtain the disease resistant plant with higher disease resistance than the target plant.

6. The method according to claim 5, wherein the reducing the content and/or the activity of the protein in the target plant is achieved by reducing an expression level and/or an activity of a coding gene of the protein in the target plant.

7. The method according to claim 6, wherein the reducing the expression level and/or the activity of the coding gene of the protein in the target plant is achieved by reducing or inactivating the activity of the coding gene of the protein in a genome of the target plant using gene mutation, gene knockout, gene editing, or gene knockdown techniques.

8. The method according to claim 7, wherein the reducing or inactivating the activity of the coding gene of the protein in the genome of the target plant using the gene editing technique is performed using a CRISPR/Cas9 system, the CRISPR/Cas9 system comprises a vector expressing a sgRNA targeting the coding gene of the protein, and target sequences of the sgRNA are SEQ ID NO: 14 and SEQ ID NO: 15.

9. A method for preparing a maize with increased disease resistance, comprising the following steps: mutating ZmLHT1 gene shown in SEQ ID NO: 1 in a maize genome into ZmLHT1/+2 bp gene, ZmLHT1/−8 bp gene, or ZmLHT1/−25 bp gene, to obtain the maize with increased disease resistance; wherein the nucleotide sequence of the ZmLHT1/+2 bp gene is as shown in SEQ ID NO: 16, the nucleotide sequence of the ZmLHT1/−8 bp gene is as shown in SEQ ID NO: 17, and the nucleotide sequence of the ZmLHT1/−25 bp gene is as shown in SEQ ID NO: 18.

10. A method for improving maize disease resistance, comprising using the ZmLHT1/+2 bp gene, the ZmLHT1/−8 bp gene, or the ZmLHT1/−25 bp gene in the method according to claim 9.

11. A disease resistant plant obtained by the method according to claim 5.

12. The disease resistant plant according to claim 11, wherein the disease resistant plant is one of the followings:
G1) monocotyledonous or dicotyledonous plants;
G2) poaceae plants;
G3) plants of genus Zea.

13. The disease resistant plant according to claim 11, wherein a disease resistance is a resistance to southern leaf blight.

14. A maize with improved disease resistance prepared by the method according to claim 9.

15. The maize according to claim 14, wherein a disease resistance is a resistance to southern leaf blight.

16. The method according to claim 3, wherein the protein is derived from maize.

17. The method according to claim 5, wherein the protein is derived from maize.

18. The disease resistant plant according to claim 11, wherein in the method for cultivating the disease resistant plant, the reducing the content and/or the activity of the protein in the target plant is achieved by reducing an expression level and/or an activity of a coding gene of the protein in the target plant.

19. The disease resistant plant according to claim 18, wherein in the method for cultivating the disease resistant plant, the reducing the expression level and/or the activity of the coding gene of the protein in the target plant is achieved by reducing or inactivating the activity of the coding gene of the protein in a genome of the target plant using gene mutation, gene knockout, gene editing, or gene knockdown techniques

20. The disease resistant plant according to claim 19, wherein in the method for cultivating the disease resistant plant, the reducing or inactivating the activity of the coding gene of the protein in the genome of the target plant using the gene editing technique is performed using a CRISPR/Cas9 system, the CRISPR/Cas9 system comprises a vector expressing a sgRNA targeting the coding gene of the protein, and target sequences of the sgRNA are SEQ ID NO: 14 and SEQ ID NO: 15.
